(19) United States
(12) Patent Application Publication
(10) Pub. No.: US 2023/0251183 A1
(43) Pub. Date: Aug. 10, 2023

(54) DETECTION KIT, DETECTION APPARATUS AND METHOD OF MANUFACTURING DETECTION KIT

(71) Applicant: Pukyong National University Industry-University Cooperation Foundation, Busan (KR)

(72) Inventors: Joong Ho SHIN, Busan (KR); Ye Lin KIM, Changwon-si (KR); Minseok KWAK, Busan (KR)

(21) Appl. No.: 18/164,240

(22) Filed: Feb. 3, 2023

(30) Foreign Application Priority Data
Feb. 4, 2022 (KR) 10-2022-0014893

(51) Int. Cl. G01N 21/17

(52) U.S. Cl. CPC G01N 21/171

(57) ABSTRACT

A detection kit, a detection apparatus and a method for manufacturing a detection kit are provided. The detection kit according to an aspect of the present invention may include a plate-shaped detection member; a first photothermal area which is formed on one side of the detection member and fixes a photothermal material that generates heat exposed to light; and a first sample area which is disposed adjacent to the first photothermal area to receive heat from the first photothermal area when light is irradiated onto the first photothermal area.

### CROSS-REFERENCE TO RELATED APPLICATION

This application claims priority to and the benefit of Korean Patent Application No. 10-2022-0014893, filed on Feb. 4, 2022, the disclosure of which is incorporated herein by reference in its entirety.

### TECHNICAL FIELD

The present invention relates to a detection kit, a detection apparatus and a method for manufacturing a detection kit. More specifically, the present invention relates to a detection kit, a detection apparatus and a method for manufacturing a detection kit, which are capable of causing a chemical reaction in an isothermal environment.

### BACKGROUND ART

Polymerase chain reaction is a technique which is capable of replicating a small amount of genetic material and amplifying the same in a large amount in a short period of time, and it is widely used in many fields such as disease diagnosis, forensic investigation and the like.

The polymerase chain reaction consists of three steps: DNA denaturation, primer annealing and DNA synthesis as one process, and each step requires different temperatures of 90 to 95° Celsius, 55 to 65° Celsius and 72° Celsius, respectively. The time required for one process is as short as 1 to 2 minutes, but since the process must be repeated 30 to 40 times, it takes more than an hour. In addition, an apparatus capable of temperature conversion is essential to adjust the temperature required for each step. However, such equipment has limitations in that it is bulky and expensive, and it is not suitable for point-of-care testing (POCT) because it is not easy to carry.

Recently, in order to overcome these limitations, isothermal amplification methods capable of replicating genetic materials at a constant temperature without temperature change have been used. Loop-mediated isothermal amplification (LAMP) with high specificity and sensitivity by using 6 primers, which is one of the isothermal amplification methods, is capable of replicating the target nucleic acid at 65° Celsius, and since it takes about 30 minutes, it takes less than half the time compared to a conventional polymerase chain reaction.

However, since the use of an additional device such as a hot plate or an external power supply device with high power consumption such as an oven is unavoidable in order to maintain a temperature of 65° Celsius, there is a limitation in that it is difficult to apply to POCT.

Accordingly, there is a demand for a detection kit capable of properly maintaining an isothermal temperature while consuming less power so as to facilitate POCT application.

### DISCLOSURE

### Technical Problem

The present invention has been devised to solve the above problems, and an object of the present invention is to provide a detection kit, a detection apparatus and a method for manufacturing a detection kit, which are capable of maintaining a sample at an isothermal temperature with low power consumption.

Another object of the present invention is to provide a detection kit, a detection apparatus and a method for manufacturing a detection kit, which are easy to use and can be maintained at low cost.

Still another object of the present invention is to provide a miniaturized and highly portable detection kit, detection apparatus and a method for manufacturing a detection kit.

The problems of the present invention are not limited to the problems mentioned above, and other problems that are not mentioned will be clearly understood by those skilled in the art from the description below.

### Technical Solution

The detection kit according to an aspect of the present invention may include a plate-shaped detection member; a first photothermal area which is formed on one side of the detection member and fixes a photothermal material that generates heat exposed to light; and a first sample area which is disposed adjacent to the first photothermal area to receive heat from the first photothermal area when light is irradiated onto the first photothermal area.

In this case, the first photothermal area may be formed in a hollow tubular shape extending from one side to the other side of the detection member, and wherein the first sample area may extend from one side to the other side of the detection member and may be formed inside the first photothermal area.

In this case, the first photothermal area may be formed in a cylindrical shape.

In this case, the detection member may be formed of a material including cellulose to have hygroscopicity.

In this case, the photothermal material may be a mixture of carbon black and poly(dimethylsiloxane).

In this case, the wavelength of light exposed to the photothermal material may be 800 nm to 1,000 nm.

In this case, the concentration of carbon black of the photothermal material may be 0.5 to 1.5%.

In this case, the photothermal material may be a mixture of gold nanoparticles and poly(dimethylsiloxane), and wherein the size of the gold nanoparticles may be 10 nm to 100 nm.

In this case, the wavelength of light exposed to the photothermal material may be 500 nm to 600 nm.

In this case, a sample may be fixed to the first sample area, and wherein the sample may include a detection material that receives heat from the first photothermal area and causes a chemical reaction at a predetermined temperature.

In this case, the sample may further include an indicator whose color changes according to pH, and wherein the detection material may be a material whose pH changes when a chemical reaction occurs.

In this case, the detection kit may further include a first protection member which is laminated on one side of the detection member; and a second protection member which is laminated on the other side of the detection member.

In this case, the first protection member may be formed to have a larger area than one surface of the detection member such that a first edge part is disposed outside the one surface of the detection member, and wherein the second protection member may be formed to have a larger area than the other surface of the detection member such that a second edge part is disposed outside the other surface of the detection member.

In this case, the first edge part of the first protection member may be bonded to the second edge part of the second protection member such that the detection member is disposed between the first protection member and the second protection member in a sealed state.

In this case, the detection kit may further include a second photothermal area which is formed on the other side of the detection member and fixes a photothermal material that generates heat exposed to light; and a second sample area which is disposed adjacent to the second photothermal area to receive heat from the second photothermal area when light is irradiated onto the second photothermal area.

In this case, the first sample fixed to the first sample area may include a detection material whose pH changes while receiving heat from the first photothermal area to cause a chemical reaction at a predetermined temperature and an indicator whose color changes according to pH; and wherein the second sample fixed to the second sample area may include an indicator whose color changes according to pH.

The detection apparatus provided with the detection kit according to an aspect of the present invention may include the above-described detection kit; an enclosure-shaped housing with a through-hole formed on one side; a holder which is detachably coupled to one surface of the housing and supported by the detection kit; a light irradiation module which is fixed to the inside of the housing and irradiates light onto the first photothermal area through the through-hole; and a power supply module which is built into the housing and supplies power to the light irradiation module.

In this case, the holder may be provided with an insertion groove into which the detection kit can be slid in one direction and inserted such that the first photothermal area of the detection kit is disposed adjacent to the through-hole.

In this case, the detection kit may further include a second photothermal area which is formed on the other side of the detection member and fixes a photothermal material that generates heat exposed to light; and a second sample area which is disposed adjacent to the second photothermal area to receive heat from the second photothermal area when light is irradiated onto the second photothermal area, and wherein the light irradiation module may include a first light irradiation member which irradiates light onto the first photothermal area; and a second light irradiation member which irradiates light onto the second photothermal area.

The method for manufacturing the detection kit according to an aspect of the present invention may include a photothermal material fixing step of fixing the photothermal material to the first photothermal area of the detection member; a punching step of removing a central portion of the first photothermal area; a first protection member attaching step of attaching a first protection member to one surface of the detection member; and a first sample area forming step of forming the first sample area at the central portion of the penetrated first photothermal area.

In this case, the photothermal material fixing step may include a photothermal material applying step of applying the photothermal material to one surface of the first photothermal area of the detection member; a photothermal material absorbing step of absorbing the photothermal material into the first photothermal area and reaching the other surface of the first photothermal area; and a photothermal material curing step of curing the photothermal material in a state where the photothermal material is absorbed in the first photothermal area.

In this case, in the first sample area forming step, the first sample area may be formed by inserting a member identical to the detection member into the central portion of the penetrated first photothermal area.

In this case, the method may further include a sample fixing step of fixing a sample to the first sample area; and a second protection member attaching step of attaching a second protection member to the other surface of the detection member. In this case, in the second protection member attaching step, a first edge part of the first protection member and a second edge part of the second protection member may be bonded such that the detection member is disposed between the first protection member and the second protection member in a sealed state.

### Advantageous Effects

The detection kit, detection apparatus and method for manufacturing a detection kit according to an exemplary embodiment of the present invention maintains a sample at an isothermal temperature through heat generated by irradiating light onto a photothermal material, thereby maintaining the sample at an isothermal temperature with low power consumption.

The detection kit, detection apparatus and method for manufacturing a detection kit according to an exemplary embodiment of the present invention include a detection member that can be used once, and thus, it can be easily used and maintained at a low cost.

The detection kit, detection apparatus and method for manufacturing a detection kit according to an exemplary embodiment of the present invention have a minimized configuration for heating a sample only with a light irradiation module, and thus, it can be used for point-of-care t ng(POCT) due to the small size and high portability.

The effects of the present invention are not limited to the above-described effects, and it should be understood to include all effects that can be inferred from the description of the present invention or the configuration of the invention described in the claims.

### DESCRIPTION OF DRAWINGS

FIG. 1 is a perspective view of the detection kit according to an exemplary embodiment of the present invention.

FIG. 2 is an exploded perspective view of the detection kit according to an exemplary embodiment of the present invention.

FIG. 3 is an enlarged cross-sectional view illustrated by magnifying the cross-section taken along the line A-A of FIG. 1.

FIG. 4 is a graph showing the power of the light irradiation module and the temperature according to the concentration of carbon black included in the photothermal material when light is irradiated to the photothermal material of the detection kit according to an exemplary embodiment of the present invention.

FIG. 5 is a graph showing the temperature of the photothermal material according to the irradiation time of light onto the photothermal material of the detection kit according to an exemplary embodiment of the present invention.

FIG. 6 is a set of views showing color changes over time in a first sample area and a second sample area of the detection kit according to an exemplary embodiment of the present invention.

FIG. 7 is a perspective view of the detection apparatus having a detection kit according to an exemplary embodiment of the present invention.

FIG. 8 is an exploded perspective view of the detection apparatus having a detection kit according to an exemplary embodiment of the present invention.

FIG. 9 is a flowchart illustrating the method for manufacturing a detection kit for manufacturing the detection kit according to an exemplary embodiment of the present invention.

FIG. 10 is a flowchart illustrating a photothermal material fixing step of the method for manufacturing a detection kit for manufacturing the detection kit according to an exemplary embodiment of the present invention.

FIG. 11 is a view showing a photothermal material fixing step of the method for manufacturing a detection kit for manufacturing the detection kit according to an exemplary embodiment of the present invention.

FIG. 12 is a view showing a punching step of the method for manufacturing a detection kit for manufacturing the detection kit according to an exemplary embodiment of the present invention.

FIG. 13 is a view showing a first protection member attaching step and a first sample area forming step of the method for manufacturing a detection kit for manufacturing the detection kit according to an exemplary embodiment of the present invention.

FIG. 14 is a view showing a sample fixing step of the method for manufacturing a detection kit for manufacturing the detection kit according to an exemplary embodiment of the present invention.

FIG. 15 is a view showing a second protection member attaching step of the method for manufacturing a detection kit for manufacturing the detection kit according to an exemplary embodiment of the present invention.

### MODES OF THE INVENTION

Hereinafter, exemplary embodiments of the present invention will be described in detail so that those of ordinary skill in the art can easily practice the present invention with reference to the accompanying drawings. The present invention may be embodied in many different forms and is not limited to the exemplary embodiments set forth herein. In the drawings, parts that are unrelated to the description are omitted for clarity, and throughout the specification, like reference numerals denote like elements.

Terms and words used in the present specification and claims should not be construed as limited to their usual or dictionary definition, and they should be interpreted as a meaning and concept consistent with the technical idea of the present invention based on the principle that inventors may appropriately define the terms and concept in order to describe their own invention in the best way.

Accordingly, the exemplary embodiments described in the present specification and the configurations shown in the drawings correspond to preferred exemplary embodiments of the present invention, and do not represent all the technical spirit of the present invention, and thus, the configurations may have various examples of equivalent and modification that can replace them at the time of filing the present invention.

It is understood that the terms “include” or “have”, when used in the present specification, are intended to describe the presence of stated features, integers, steps, operations, elements, components and/or a combination thereof, but do not preclude the possibility of the presence or addition of one or more other features, integers, steps, operations, elements, components or a combination thereof.

The presence of an element in/on “front”, “rear”, “upper or above or top” or “lower or below or bottom” of another element includes not only being disposed in/on “front”, “rear”, “upper or above or top” or “lower or below or bottom” directly in contact with other elements, but also cases in which another element being disposed in the middle, unless otherwise specified. In addition, unless otherwise specified, that an element is “connected” to another element includes not only direct connection to each other but also indirect connection to each other.

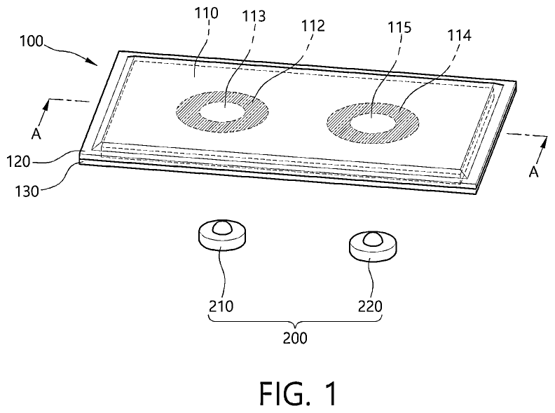

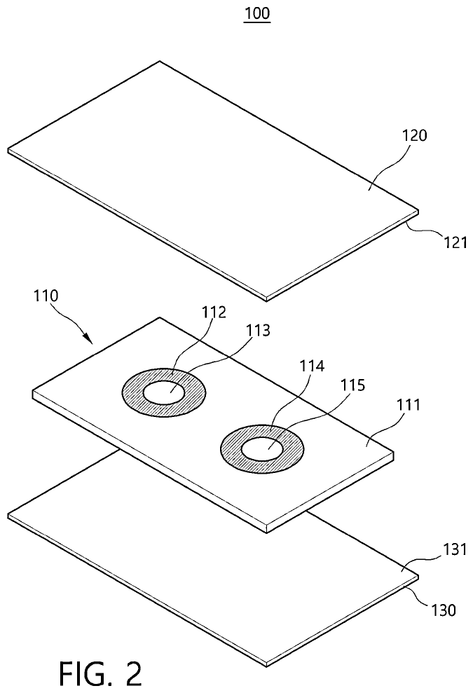

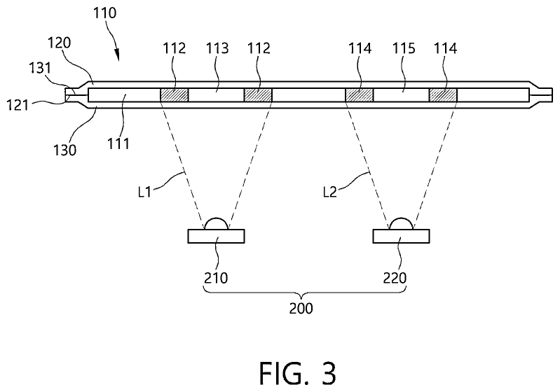

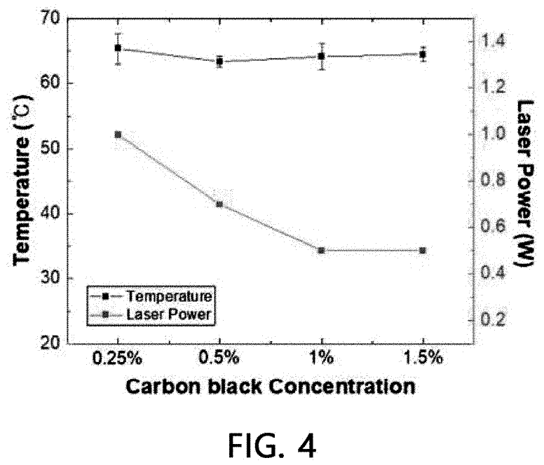

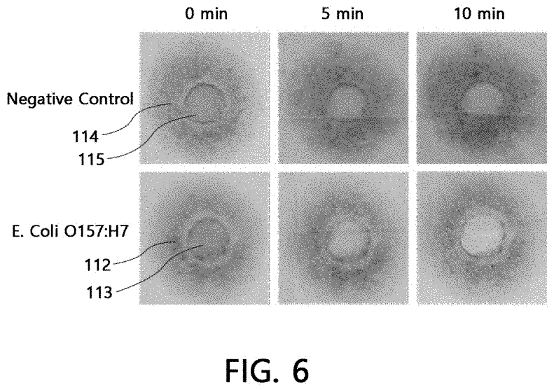

Hereinafter, the detection kit, detection apparatus and method for manufacturing a detection kit according to an exemplary embodiment of the present invention will be described with reference to the drawings. FIG. 1 is a perspective view of the detection kit according to an exemplary embodiment of the present invention. FIG. 2 is an exploded perspective view of the detection kit according to an exemplary embodiment of the present invention. FIG. 3 is an enlarged cross-sectional view illustrated by magnifying the cross-section taken along the line A-A of FIG. 1. FIG. 4 is a graph showing the power of the light irradiation module and the temperature according to the concentration of carbon black included in the photothermal material when light is irradiated to the photothermal material of the detection kit according to an exemplary embodiment of the present invention. FIG. is a graph showing the temperature of the photothermal material according to the irradiation time of light onto the photothermal material of the detection kit according to an exemplary embodiment of the present invention. FIG. 6 is a set of views showing color changes over time in a first sample area and a second sample area of the detection kit according to an exemplary embodiment of the present invention.

As illustrated in FIG. 1, the detection kit 100 according to an exemplary embodiment of the present invention may include a detection member 110, a first photothermal area 112, a first sample area 113, and a first protection member 120 and a second protection member 130.

The detection member 110 is formed in a plate shape. As illustrated in FIG. 1, the detection member 110 may be formed in a rectangular plate shape having four sides.

The detection member 110 is formed of a material having hygroscopicity. A material constituting the detection member 110 may include cellulose. That is, the detection member 110 may be formed of cellulose paper. However, the detection member 110 is not limited to a material as long as it can be formed of a material having hygroscopicity.

As illustrated in FIG. 2, a first photothermal area 112 is formed in the detection member 110. The first photothermal area 112 is formed on one side of the detection member 110.

The first photothermal area 112 includes a photothermal material. In this case, there is no limitation on how the photothermal material is included in the first photothermal area 112. For example, the first photothermal area 112 may be designated on one side of the detection member 110, and the photothermal material X1 may be absorbed and fixed to the designated first photothermal area 112. Alternatively, the first photothermal area 112 may be formed by forming with the photothermal material and attaching the first photothermal area 112 to the detection member 110.

In this case, the shape of the first photothermal area 112 is not limited. For example, as illustrated in FIGS. 2 and 3, it may be formed in a hollow tubular shape. In the present exemplary embodiment, it will be described that the first photothermal area 112 is formed in a cylindrical shape.

The first photothermal area 112 formed in a cylindrical shape is formed by extending from one side to the other side of the detection member 110. That is, one end is exposed to one surface of the detection member 110, and the other end is exposed to the other surface of the detection member 110. Accordingly, even if light is irradiated onto the first photothermal area 112 from any surface of the detection member 110, the photothermal material X1 may be exposed to light such that the detection can be performed even if the user does not have to distinguish the front side or the back side.

Since the first photothermal area 112 is formed in a hollow tubular shape, a first sample area 113 to be described below may be formed at the central portion of the first photothermal area. That is, as illustrated in FIGS. 2 and 3, the first sample area 113 is formed in a cylindrical shape filling the hollow area of the cylindrical first photothermal area 112. In this case, the first sample area 113 is formed such that the same thickness as the first photothermal area 112, that is, the distance from one side to the other side of the detection member 110 becomes the height of the cylinder. Accordingly, the first photothermal area 112, the first sample area 113 and the other detection members 110 are formed to have the same thickness. This increases the adhesion efficiency of the first protection member 120 and the second protection member 130 to be described below.

In this case, the photothermal material fixed to the first photothermal area 112 in the present specification is defined as a material that generates heat when exposed to light at a predetermined wavelength or a material including the same. The material that generates heat when exposed to light may be carbon black or gold nanoparticles, but there is no limitation as long as it is a material that generates heat when exposed to light. In the present exemplary embodiment, it will be described that the photothermal material includes a material that generates heat exposed to light.

When the photothermal material includes carbon black, the wavelength of light irradiated to the photothermal material may be 800 nm to 1,000 nm. In this case, preferably, heat may be generated from the carbon black by irradiating light having a wavelength of 808 nm.

When the photothermal material includes gold nanoparticles, the size of the gold nanoparticles used as the photothermal material may be 10 nm to 100 nm. In this case, the wavelength of light irradiated to the photothermal material may be 500 nm to 600 nm or less, and preferably, heat may be generated from the gold nanoparticles by irradiating light having a wavelength of 532 nm.

The photothermal material further includes poly(dimethylsiloxane) to be fixed to the first photothermal area 112. Poly(dimethylsiloxane) is cured together with carbon black or gold nanoparticles while being absorbed by the first photothermal area 112 such that the carbon black or gold nanoparticles can be fixed to the first photothermal area 112. Hereinafter, the photothermal material will be described as a mixture of carbon black and poly(dimethylsiloxane).

As illustrated in FIG. 3, when the first photothermal area 112 is irradiated with a laser L1 by the first light irradiation member 210, the temperature of the first photothermal area 112 rises due to the heat generated from the photothermal material. In this case, the concentration of carbon black of the photothermal material may be 0.5 to 1.5%, and preferably, 1%. This is to optimize power efficiency as illustrated in FIG. 4. More specifically, in a state where the photothermal material maintains the same temperature, power consumption of the first light irradiation member 210 decreases until the concentration of carbon black reaches 1%, but this is because there is no change in power consumption of the first light irradiation member 210 when the concentration of carbon black is more than 1%.

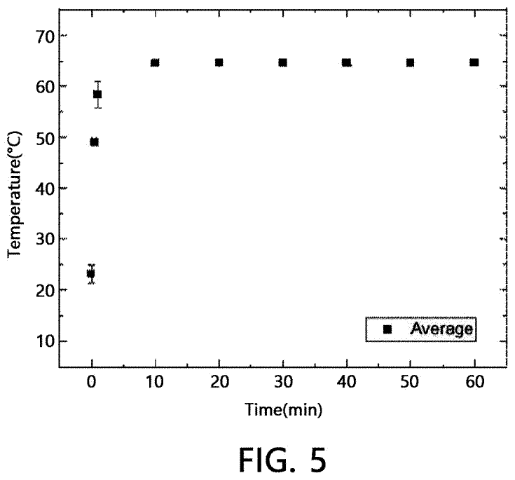

In order to maintain the first photothermal area 112 at a predetermined temperature by radiating light from the first light irradiation member 210, the time required to irradiate the photothermal material may vary as needed. For example, as illustrated in FIG. 5, in the case of a photothermal material including carbon black, the same temperature may be maintained when heated for 10 minutes or more, and the same temperature may be maintained according to the type of sample to be described below such that it is possible to induce a chemical reaction of the sample.

As illustrated in FIG. 2, the first sample area 113 is formed adjacent to the first photothermal area 112. Accordingly, the heat generated in the first photothermal area 112 is transferred.

In this case, the first sample area 113 is formed of a hygroscopic material. In addition, it may be formed of the same material as the detection member 110. In this case, the sample is absorbed and fixed to the first sample area 113. The sample absorbed and fixed to the first sample area 113 includes a detection material that receives heat from the first photothermal area 112 and causes a chemical reaction at a predetermined temperature.

As shown in FIG. 5, the detection material may be a material that causes a chemical reaction in the process of maintaining the first photothermal area 112 at 65° Celsius through the first light irradiation member 210. In this case, the chemical reaction may be the replication of nucleic acid by isothermal amplification (loop-mediated isothermal amplification).

Meanwhile, when the detection material is a material whose pH changes during a chemical reaction, the sample may further include an indicator whose color changes according to the pH. Through this, as illustrated in FIG. 6, whether the chemical reaction of the detection material is completed may be recognized by the color change of the indicator.

The shape of the first photothermal area 112 is not limited as long as it can constantly receive heat from the first sample area 113. As described above, when the first photothermal area 112 is formed in a hollow cylindrical shape, it may be formed in a tubular shape disposed inside the first photothermal area 112. Since the first photothermal area 112 is formed in a tubular shape and the first sample area 113 is formed in a cylindrical shape, the heat generated at the first photothermal area by irradiating light onto the first photothermal area 112 may be uniformly transferred to the first sample area 113.

The first sample area 113 may be integrally formed with the detection member 110 or may be coupled to the detection member 110 in a state of being formed separately. When the first sample area 113 is formed adjacent to and coupled to the first photothermal area 112 in a state where it is formed separately from the detection member 110, as illustrated in FIG. 2, the first sample area 113 may be provided with a first protection member 120 to be fixed adjacent to the photothermal area 112.

In this case, the first protection member 120 may be stacked on one surface of the detection member 110 to support the detection member 110 and the first sample area 113. An adhesive material may be applied to one surface of the first protection member 120 that is in contact with one surface of the detection member 110.

In this case, the first protection member 120 is formed in a shape similar to that of the detection member 110. For example, as illustrated in FIG. 2, when the detection member 110 is formed in a rectangular plate shape, the first protection member 120 may also be formed in a rectangular plate shape.

The first protection member 120 may be formed to have an adhesive area larger than one surface of the detection member 110. Accordingly, the user may easily attach the first protection member 120 to the detection member 110.

As illustrated in FIG. 2, a second protection member 130 may be stacked on the other surface of the detection member 110. Accordingly, the second protection member 130 is disposed to face the first protection member 120 with the detection member 110 interposed therebetween. The second protection member 130 is formed in the same manner as the first protection member 120, and the description thereof is replaced with the description of the first protection member 120.

The second protection member 130 and the first protection member 120 are formed to have a larger bonding area than the detection member 110. In this case, as illustrated in FIGS. 1 and 3, the first edge part 121 and the second edge part 131, which are edge parts of the first protection member 120 and the second protection member 130, are disposed on one surface and the outer side of the other surface of the detection member 110.

The first edge part 121 of the first protection member 120 and the second edge part 131 of the second protection member 130 are directly bonded from the outside of the detection member 110. Accordingly, the detection member 110 is disposed in an enclosed space between the first protection member 120 and the second protection member 130. Through this, it is possible to prevent the inflow of foreign matter or the evaporation of the sample during the chemical reaction of the sample in the first sample area 113, thereby increasing the reliability of the detection.

As illustrated in FIGS. 1 and 2, the detection kit 100 according to an exemplary embodiment of the present invention may further include a second photothermal area 114 and a second sample area 115. However, since the second photothermal area 114 and the second sample area 115 are the same as the first photothermal area 112 and the first sample area 113, the overlapping descriptions of the second photothermal area 114 and the second sample area will be omitted below.

As illustrated in FIG. 2, the second photothermal area 114 and the second sample area 115 are spaced apart from the first photothermal area 112 and the first sample area 113 and are arranged side by side.

In this case, as illustrated in FIG. 2, the sample is also fixed to the second sample area 115 disposed inside the second photothermal area 114. In this case, a sample including a detection material that causes a chemical reaction is fixed to the first sample area 113, and a sample that does not include the detection material is fixed to the second sample area 115. Accordingly, it is possible to determine whether the color change in the first sample area 113 is due to a foreign substance other than the detection material or a chemical reaction of the detection material by the indicator. That is, the second sample area 115 is utilized as a control to increase the accuracy of the detection.

Referring to FIG. 6, in the first sample area 113 including the detection material, the color change of the indicator may be confirmed according to the lapse of light irradiation time, whereas in the second sample area 115, it can be confirmed that there is no color change of the indicator according to the lapse of light irradiation time. Through this, it is possible to confirm that the chemical reaction of the detection material has been performed.

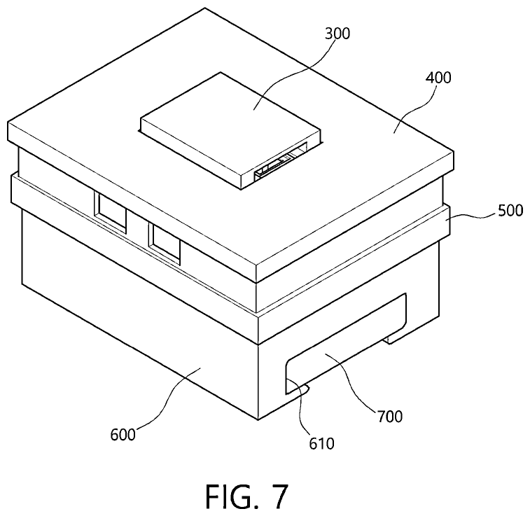

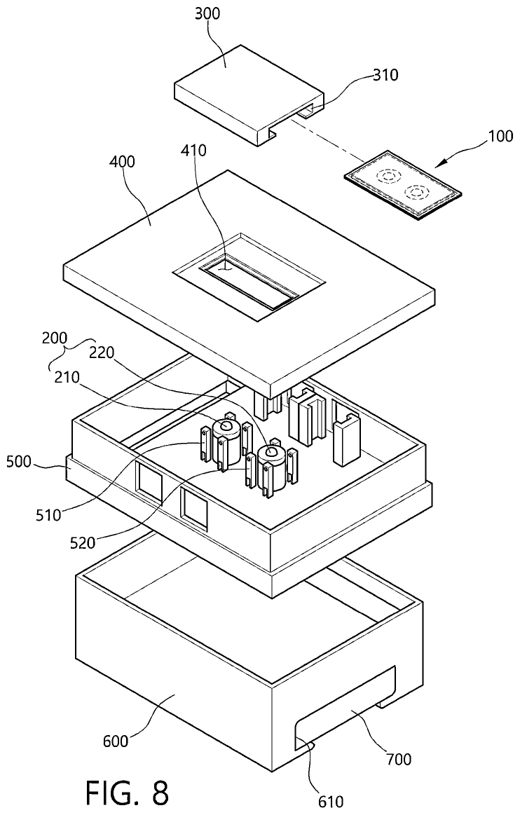

FIG. 7 is a perspective view of the detection apparatus having a detection kit according to an exemplary embodiment of the present invention. FIG. 8 is an exploded perspective view of the detection apparatus having a detection kit according to an exemplary embodiment of the present invention.

As illustrated in FIG. 7, the detection apparatus having the detection kit 100 according to an exemplary embodiment of the present invention includes the detection kit 100 described above, housings 400, 500, 600, a holder 300, a light irradiation module 200 and a power supply module 700. In this case, the description of the detection kit 100 is replaced with the above description.

The housings 400, 500, 600 are formed in a box shape. The housings 400, 500, 600 are not limited in shape as long as they can contain the light irradiation module 200 and the power supply module 700 described below. In the present exemplary embodiment, as illustrated in FIG. 8, the housings 400, 500, 600 may include a first housing 500 with a space formed therein and one side open, a cover for covering the open side of the first housing 500 and a second housing 600 coupled to the other side of the first housing 500.

In this case, the light irradiation module 200 is fixed inside the first housing 500. The light irradiation module 200 may include the number of light irradiation members corresponding to the number of photothermal areas and sample areas in the detection kit 100. For example, as illustrated in FIG. 8, the detection kit 100 may be provided with a first photothermal area 112 and a second photothermal area 114, and the first light irradiation member 210 and the second light irradiation member 220 may be provided to correspond thereto.

The first housing 500 may be provided a first support member 510 and a second support member 520 inside to support the first light irradiation member 210 and the second light irradiation member 220. The fixing method of the first support member 510 and the second support member 520 is not limited as long as the first light irradiation member 210 and the second light irradiation member 220 can be fixed. For example, as illustrated in FIG. 8, it may be formed in a plurality of pillar shapes disposed along outer circumferential surfaces of the first light irradiation member 210 and the second light irradiation member 220.

A through-hole 410 is formed on the cover 400 coupled to the open side of the first housing 500. The light irradiation module 200 may irradiate light onto the detection kit 100 through the through-hole 410.

A holder is coupled to the side of the through-hole 410 of the cover 400. The holder 300 serves to support the detection kit 100. In this case, the holder 300 is detachably coupled to the cover 400. Through this, the holder 300 may be easily coupled to the cover 400 while the detection kit 100 is coupled to the holder.

The holder 300 is formed with an insertion groove 310 into which the detection kit 100 can be inserted by sliding in the lateral direction. The insertion groove 310 extends along both ends of the detection kit 100 to support both ends of the detection kit 100. In this case, when the holder 300 is coupled to the cover 400 in a state where the detection kit 100 is inserted by sliding on the holder 300, as illustrated in FIG. 3, the lasers L1, L2 which are irradiated by the first light irradiation member 210 and the second light irradiation member 220 are positioned to be irradiated to the first photothermal area 112 and the second photothermal area 114 of the detection kit 100, respectively.

The light irradiation module 200 may use a component capable of irradiating light having different wavelengths according to photothermal materials. In this case, various known components may be used in the light irradiation module 200, and the components used are not limited.

The second housing 600 is coupled to the other surface of the first housing 500, which is an opposite surface to the surface on which the cover 400 is installed. A power supply module 700 for supplying power to the light irradiation module 200 is embedded in the second housing 600.

The power supply module 700 may be detachably coupled to the second housing 600. In this case, as illustrated in FIG. 8, the power supply module 700 may be inserted and fixed to the outside of the second housing 600 so as to universally utilize various types of well-known batteries. To this end, a power supply module insertion groove 610 of the power supply module 700 may be formed on one side of the second housing 600.

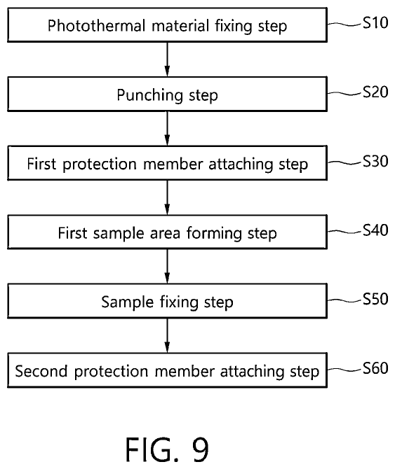

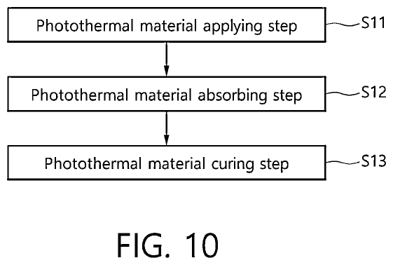

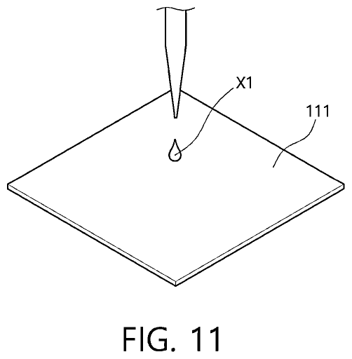

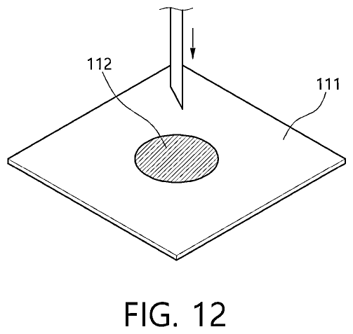

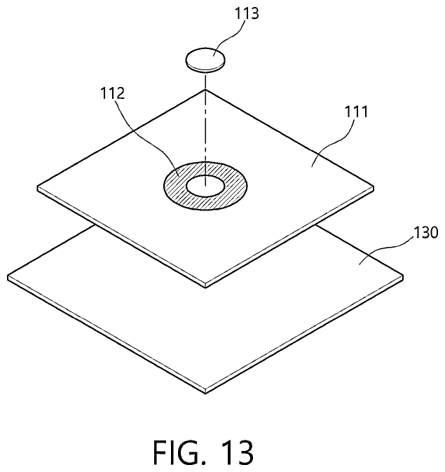

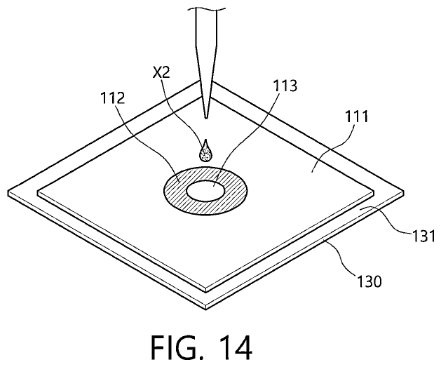

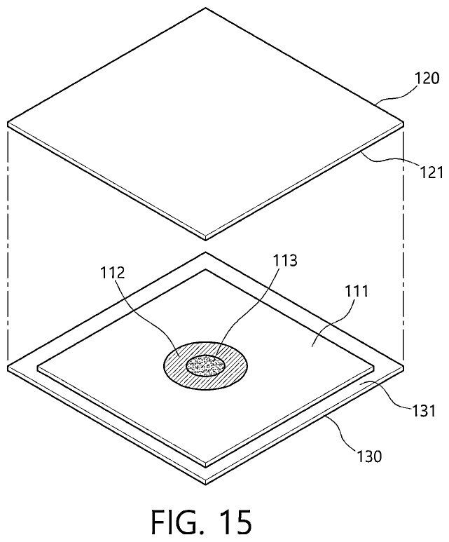

Hereinafter, the method for manufacturing a detection kit for manufacturing the detection kit according to an exemplary embodiment of the present invention will be described with reference to FIGS. 9 to 15. In this case, contents overlapping with the above-described contents are omitted. FIG. 9 is a flowchart illustrating the method for manufacturing a detection kit for manufacturing the detection kit according to an exemplary embodiment of the present invention. FIG. 10 is a flowchart illustrating a photothermal material fixing step of the method for manufacturing a detection kit for manufacturing the detection kit according to an exemplary embodiment of the present invention. FIG. 11 is a view showing a photothermal material fixing step of the method for manufacturing a detection kit for manufacturing the detection kit according to an exemplary embodiment of the present invention. FIG. 12 is a view showing a punching step of the method for manufacturing a detection kit for manufacturing the detection kit according to an exemplary embodiment of the present invention. FIG. 13 is a view showing a first protection member attaching step and a first sample area forming step of the method for manufacturing a detection kit for manufacturing the detection kit according to an exemplary embodiment of the present invention. FIG. 14 is a view showing a sample fixing step of the method for manufacturing a detection kit for manufacturing the detection kit according to an exemplary embodiment of the present invention. FIG. 15 is a view showing a second protection member attaching step of the method for manufacturing a detection kit for manufacturing the detection kit according to an exemplary embodiment of the present invention.

As illustrated in FIG. 9, the method for manufacturing a detection kit for manufacturing the detection kit according to an exemplary embodiment of the present invention includes a photothermal material fixing step S10, a punching step S20, a first protection member attaching step S30, a first sample area forming S40, a sample fixing S50 and a second protection member attaching step S60.

In the photothermal material fixing step S10, the photothermal material is fixed to the position of the first photothermal area 112 of the plate-shaped detection member 110. To this end, as illustrated in FIG. 10, the photothermal material fixing step S10 includes a photothermal material applying step S11, a photothermal material absorbing step S12 and a photothermal material curing step S13.

In the photothermal material applying step S11, as illustrated in FIG. 11, the photothermal material X1 is applied to one surface of the first photothermal area 112 of the detection member 110.

In the photothermal material absorbing step S12, the applied photothermal material X1 is absorbed in the first photothermal area 112 to wait for a predetermined time to move to the other surface. Accordingly, the photothermal material X1 is absorbed in a cylindrical shape in the first photothermal area 112.

In the photothermal material curing step S13, the detection member 110 is cured in a state where the photothermal material X1 is absorbed in the first photothermal area 112. In this case, there is no limitation on the method of curing the detection member 110. For example, it may be cured by placing in an oven and heating the same. In this case, poly(dimethylsiloxane) included in the photothermal material X1 is cured in the first photothermal area 112 of the cured detection member 110, and carbon black included in the photothermal material X1 is fixed to the first photothermal area 112.

In the punching step S20, as illustrated in FIG. 12, the central portion of the first photothermal area 112 is removed through punching. In this case, the central portion of the first photothermal area 112 is circularly penetrated. The size of the central portion of the penetrated first photothermal area 112 may be formed differently as needed. In the present exemplary embodiment, the central diameter of the first photothermal area 112 is 3 mm.

In the first protection member attaching S30, as illustrated in FIG. 13, the first protection member 120 is attached to one surface of the detection member 110. In this case, as illustrated in FIG. 14, the first protection member 120 is bonded to one surface of the detection member 110 such that the first edge part 121 of the first protection member 120 may be disposed outside the detection member 110.

In the first sample area forming step S40, a circular member made of the same material as the detection member 110 is inserted to form the first sample area 113 at the central portion of the penetrated first photothermal area 112. In this case, one surface of the tubular member is bonded to the first protection member 120 to form the first sample area 113.

In the sample fixing step S50, as illustrated in FIG. 14, the sample X2 is fixed by dropping and absorbing the sample into the first sample area 113.

In the second protection member attaching step S60, the second protection member 130 is attached to the other surface of the detection member 110. In this case, as illustrated in FIG. 15, the second edge part 131 of the second protection member 130 is disposed on the outer side of the detection member 110 such that the first edge part 121 of the first protection member 120 and the second edge part 131 of the second protection member 130 can be bonded. By bonding the first edge part 121 and the second edge part 131 of the first protection member 120 and the second protection member 130, the detection member 110 is in a sealed state between the first protection member 120 and the second protection member.

Although the detection kit, detection apparatus and method for manufacturing a detection kit according to an exemplary embodiment of the present invention have been described above, it will be clearly understood by one of ordinary skill in the art to which the present invention pertains that the detection kit according to the present exemplary embodiment can be used not only for the replication of nucleic acid, but also it can be used as a detection kit for confirming whether a reaction has occurred by performing a chemical reaction by maintaining an isothermal temperature.

As described above, the preferred exemplary embodiments according to the present invention have been reviewed, and the fact that the present invention can be embodied in other specific forms in addition to the above-described exemplary embodiments without departing from the spirit or scope thereof is apparent to those of ordinary skill in the art. Therefore, the foregoing exemplary embodiments are to be regarded as illustrative rather than restrictive, and thus, the present invention is not limited to the foregoing description, but may be modified within the scope of the appended claims and their equivalents.

[Explanation of Reference Numerals]

100: Detection kit
110: Detection member
112: First photothermal area
113: First sample area
114: Second photothermal area
115: Second sample area
120: First protection member
121: First edge part
130: Second protection member
131: Second edge part
200: Light irradiation module
210: First light irradiation member
220 Second light irradiation member
300: Holder
310: Insertion groove
400: Cover
410: Through-hole
500: First housing
510: First support member
520: Second support member
600: Second housing
610: Power supply module insertion groove
700: Power supply module

1. A detection kit, comprising:
a plate-shaped detection member;
a first photothermal area which is formed on one side of the detection member and fixes a photothermal material that generates heat exposed to light; and
a first sample area which is disposed adjacent to the first photothermal area to receive heat from the first photothermal area when light is irradiated onto the first photothermal area.

2. The detection kit of claim 1, wherein the first photothermal area is formed in a hollow tubular shape extending from one side to the other side of the detection member, and
wherein the first sample area extends from one side to the other side of the detection member and is formed inside the first photothermal area.

3. The detection kit of claim 2, wherein the first photothermal area is formed in a cylindrical shape.

4. The detection kit of claim 1, wherein the detection member is formed of a material comprising cellulose to have hygroscopicity.

5. The detection kit of claim 1, wherein the photothermal material is a mixture of carbon black and poly(dimethylsiloxane).

6. The detection kit of claim 5, wherein the wavelength of light exposed to the photothermal material is 800 nm to 1,000 nm.

7. The detection kit of claim 5, wherein the concentration of carbon black of the photothermal material is 0.5 to 1.5%.

8. The detection kit of claim 1, wherein the photothermal material is a mixture of gold nanoparticles and poly(dimethylsiloxane), and
wherein the size of the gold nanoparticles is 10 nm to 100 nm.

9. The detection kit of claim 8, wherein the wavelength of light exposed to the photothermal material is 500 nm to 600 nm.

10. The detection kit of claim 1, wherein a sample is fixed to the first sample area, and
wherein the sample comprises a detection material that receives heat from the first photothermal area and causes a chemical reaction at a predetermined temperature.

11. The detection kit of claim 10, wherein the sample further comprises an indicator whose color changes according to pH, and
wherein the detection material is a material whose pH changes when a chemical reaction occurs.

12. The detection kit of claim 1, further comprising:
a first protection member which is laminated on one side of the detection member; and
a second protection member which is laminated on the other side of the detection member.

13. The detection kit of claim 12, wherein the first protection member is formed to have a larger area than one surface of the detection member such that a first edge part is disposed outside the one surface of the detection member, and
wherein the second protection member is formed to have a larger area than the other surface of the detection member such that a second edge part is disposed outside the other surface of the detection member.

14. The detection kit of claim 13, wherein the first edge part of the first protection member is bonded to the second edge part of the second protection member such that the detection member is disposed between the first protection member and the second protection member in a sealed state.

15. The detection kit of claim 1, further comprising:
a second photothermal area which is formed on the other side of the detection member and fixes a photothermal material that generates heat exposed to light; and
a second sample area which is disposed adjacent to the second photothermal area to receive heat from the second photothermal area when light is irradiated onto the second photothermal area.

16. The detection kit of claim 15, wherein the first sample fixed to the first sample area comprises a detection material whose pH changes while receiving heat from the first photothermal area to cause a chemical reaction at a predetermined temperature and an indicator whose color changes according to pH; and
wherein the second sample fixed to the second sample area comprises an indicator whose color changes according to pH.

17. A detection apparatus, comprising:
the detection kit according to claim 1;
an enclosure-shaped housing with a through-hole formed on one side;
a holder which is detachably coupled to one surface of the housing and supported by the detection kit;
a light irradiation module which is fixed to the inside of the housing and irradiates light onto the first photothermal area through the through-hole; and
a power supply module which is built into the housing and supplies power to the light irradiation module.

18. The detection apparatus of claim 17, wherein the holder is provided with an insertion groove into which the detection kit can be slid in one direction and inserted such that the first photothermal area of the detection kit is disposed adjacent to the through-hole.

19. The detection apparatus of claim 17, wherein the detection kit further comprises:
a second photothermal area which is formed on the other side of the detection member and fixes a photothermal material that generates heat exposed to light; and
a second sample area which is disposed adjacent to the second photothermal area to receive heat from the second photothermal area when light is irradiated onto the second photothermal area, and
wherein the light irradiation module comprises:
a first light irradiation member which irradiates light onto the first photothermal area; and
a second light irradiation member which irradiates light onto the second photothermal area.

20. A method for manufacturing a detection kit which manufactures the detection kit according to claim 1, comprising:
a photothermal material fixing step of fixing the photothermal material to the first photothermal area of the detection member;
a punching step of removing a central portion of the first photothermal area;
a first protection member attaching step of attaching a first protection member to one surface of the detection member; and
a first sample area forming step of forming the first sample area at the central portion of the penetrated first photothermal area.

21. The method of claim 20, wherein the photothermal material fixing step comprises:
a photothermal material applying step of applying the photothermal material to one surface of the first photothermal area of the detection member;
a photothermal material absorbing step of absorbing the photothermal material into the first photothermal area and reaching the other surface of the first photothermal area; and
a photothermal material curing step of curing the photothermal material in a state where the photothermal material is absorbed in the first photothermal area.

22. The method of claim 20, wherein in the first sample area forming step, the first sample area is formed by inserting a member identical to the detection member into the central portion of the penetrated first photothermal area.

23. The method of claim 20, further comprising:
a sample fixing step of fixing a sample to the first sample area; and
a second protection member attaching step of attaching a second protection member to the other surface of the detection member.

24. The method of claim 23, wherein in the second protection member attaching step, a first edge part of the first protection member and a second edge part of the second protection member are bonded such that the detection member is disposed between the first protection member and the second protection member in a sealed state.
